(19) United States
(12) Patent Application Publication
(10) Pub. No.: US 2020/0046768 A1
(43) Pub. Date: Feb. 13, 2020

(54) DOUBLE NEGATIVE T CELLS AND PD-1 BLOCKADE FOR THE TREATMENT OF CANCER

(71) Applicant: University Health Network, Toronto (CA)

(72) Inventors: Li Zhang, Toronto (CA); Dalam Ly, Toronto (CA); Linan Fang, Toronto (CA)

(21) Appl. No.: 16/536,948

(22) Filed: Aug. 9, 2019

Related U.S. Application Data
(60) Provisional application No. 62/716,665, filed on Aug. 9, 2018.

(51) Int. Cl. A61K 35/17; C07K 16/28; A61P 35/00

(52) U.S. Cl. CPC A61K 35/17; A61P 35/00; C07K 16/2827; C07K 16/2818

(57) ABSTRACT

Methods for the treatment of cancer using double negative T cells (DNTs) and a PD-1 or PD-L1 inhibitor are described. Tumors treated with DNTs and a PD-1 inhibitor exhibited increased DNT cell infiltration and increased cytotoxicity towards non-small cell lung cancer (NSCLC) cells. Also described are compositions and kits comprising DNTs and a PD-1 or PD-L1 inhibitor and their use for the treatment of cancer.

### RELATED APPLICATIONS

This application claims priority to U.S. Provisional Patent Application No. 62/716,665 filed Aug. 9, 2018 the entire contents of which are hereby incorporated by reference.

### FIELD

The disclosure relates to immunotherapy for the treatment of cancer and more specifically to immunotherapy for the treatment of cancer using the combination of double negative T cells and a PD-1 or PD-L1 inhibitor.

### BACKGROUND OF THE INVENTION

The natural tumor-killing properties of CD4− CD8−double negative T (DNT) cells provide a tremendous opportunity for the treatment of cancer. DNT cell therapy has been demonstrated to be a promising approach for treatment of acute myeloid leukemia (AML) (1-3). In those studies, a protocol was established allowing for ex vivo expansion of therapeutic numbers of DNTs with high purity from healthy donors (1). The ex vivo expanded DNTs from healthy donors can effectively lyse primary AML cells without observed toxicity towards normal cells and tissues (1). Though many advances have been made with respect to the characterization and development of DNT cells for use in the treatment of hematopoietic malignancies, challenges remain in adopting their use for treatment of solid tumors. Besides differences in etiology, solid tumors, such as melanoma and lung cancers develop in niches that differ from hematopoietic compartments. Primary tumor sites have established an immune-privileged tumor microenvironment (TME) that is generally hostile to cytotoxic T cells (4). The need to overcome this immune hostile microenvironment has been the main challenge for adoptive cell therapies (ACT) for solid tumors (4, 5).

One mechanism by which tumors promote immune suppression is through engagement of immune checkpoint receptors which regulate T cell activity (5). One such checkpoint, termed PD-1 and PD-L1 has been implicated in tumor immunoevasion of many cancers (6). Programmed cell death 1 (PD-1) is a surface protein expressed on activated T cells that mediate inhibitory signals upon engagement of its ligand PD-L1. Though PD-L1 is also expressed on other hematopoietic cells, its expression on tumor epithelial cells implicates its role in cancer immunoevasion (6). Indeed, blocking antibodies targeting this pathway have lead clinical reduction of tumor growth and patient overall survival, with mechanisms attributed to increases in the expansion and activity of resident anti-tumor T cells (7). The clinical success of anti-PD-1 therapy has led to significant development of a new class of drugs termed immune checkpoint blockade (ICB). Combinations of ICB with additional therapies that directly target tumor such as chemotherapy are currently being tested (8-9). Whether ICB therapy in combination with DNTs can be used to target solid tumors remains unclear and additional treatment options for cancers such as lung are needed.

### SUMMARY

In one aspect, it has been determined that the combination of double negative T cells (DNTs) with PD-1 blockade enhances DNT treatment efficacy by increasing DNT cell infiltration in solid tumor.

To determine the efficacy of ex vivo DNT cell therapy on lung cancer, NSG mice subcutaneously engrafted with human lung cancer cell line H460 were systemically infused with DNT cells or DNT cell with Nivolumab in three doses when tumor size reached 100 mm³, the tumor size and DNT cell infiltration level were monitored.

As demonstrated in the Examples, xenograft mice treated with DNT cells alone reduced tumor growth by 38±16%. Surprisingly, a greater reduction (65±20%) was observed when DNT cell treatment was combined with PD-1 blockade, whereas PD-1 blockade alone had no significant effect. Compared to DNT cell transfer alone, DNT cells with PD-1 blockade led to a greater infiltration of DNT cells with high expression of cytotoxicity markers NKG2D, DNAM-1 and perforin and reduced inhibitory markers TIM3 and LAG3.

Accordingly, in one embodiment there is provided a composition comprising double negative T cells (DNTs) and a PD-1 or PD-L1 inhibitor. In one embodiment, the PD-1 or PD-L1 inhibitor is an antibody such as Nivolumab. In one embodiment, the DNTs have the phenotype CD4−, CD8−, CD3+, γδ-TCR+ and/or αβ-TCR. In one embodiment, the composition further comprises a pharmaceutically acceptable carrier.

In one embodiment, there is provided a kit comprising double negative T cells (DNTs) and a PD-1 or PD-L1 inhibitor. Optionally, the DNTs and the PD-1 or PD-L1 inhibitor are in the same container, such as in a composition, or in separate containers.

The combination of DNTs and a PD-1 or PD-L1 inhibitor as described herein have a number of characteristics that make the combination useful for the treatment of cancer. For example, in one embodiment, the PD-1 or PD-L1 inhibitor increases DNT-mediated anti-tumor activity. In one embodiment, the PD-1 or PD-L1 inhibitor increases DNT tumor infiltration.

Also provided is the use of a composition or kit as described herein comprising DNTs and a PD-1 or PD-L1 inhibitor for the treatment of cancer in a subject in need thereof. In one embodiment, the cancer is melanoma or lung cancer, optionally non-small cell lung cancer (NSCLC).

In one embodiment, there is provided a method of treating cancer in a subject in need thereof, the method comprising administering to the subject double negative T cells (DNTs) and a PD-1 or PD-L1 inhibitor. Also provided is the use of DNTs and a PD-1 or PD-L1 inhibitor for treating cancer in a subject in need thereof.

In one embodiment, the PD-1 or PD-L1 inhibitor is an antibody or a fragment of antibody such as Fab, F(ab)², or single-chain variable fragment (scFv). In one embodiment, the PD-1 or PD-L1 inhibitor is a nanobody or a fusion protein.

In one embodiment, the PD-1 inhibitor is Nivolumab, Pembrolizumab, Cemiplimab, Spartalizumab, Tislelizumab, Sintilimab, Toripalimab, Camrelizumab, Dostarlimab, MGA012, or AB122. In one embodiment, the PD-L1 inhibitor is Atezolizumab, Durvalumab, Avelumab, Cosibelimab, Envafolimab or KN035.

In some embodiments, the DNTs and the PD-1 or PD-L1 inhibitor are for use or administration at the same time or at different times. In one embodiment, the DNTs are for use or administration to the subject within 60 days of the administration or use of the PD-1 or PD-L1 inhibitor. In one embodiment, the DNTs are for use or administration to the subject within 30 days of the administration or use of the PD-1 or PD-L1 inhibitor. In one embodiment, the PD-1 or PD-L1 inhibitor is for use or administration prior to the use or administration of the DNTs.

In one embodiment, the method comprises the use or administration of only a single dose of DNTs and one or more doses of the PD-1 or PD-L1 inhibitor.

In one embodiment, a first dose of DNTs and one or more subsequent doses of DNTs are for use or administered to the subject for the treatment of cancer. In one embodiment, a first dose and a second dose of DNTs are for use or administered to the subject. In one embodiment, the second dose is for use or administered to the subject within 6 months of the first dose.

In one embodiment, a first dose of a PD1 or PD-L1 inhibitor and one or more subsequent doses of a PD1 or PD-L1 inhibitor are for use or administered to the subject for the treatment of cancer. In one embodiment, a first dose and a second dose of a PD1 or PD-L1 inhibitor are for use or administered to the subject. In one embodiment, the second dose is for use or administered to the subject within 6 weeks of the first dose.

In one embodiment, the DNTs and the PD-1 or PD-L1 inhibitor are for use or administration by the same route of administration. In another embodiment, the DNTs and the PD-1 or PD-L1 inhibitor are for use or administration by different routes of administration. In one embodiment, the PD-1 or PD-L1 inhibitor increases DNT-mediated anti-tumor activity. In one embodiment, the PD-1 or PD-L1 inhibitor increases DNT tumor infiltration. In one embodiment, the PD-1 or PD-L1 inhibitor increases DNT-mediated cytotoxicity. In one embodiment, the cancer is melanoma or lung cancer. In one embodiment, the cancer is non-small cell lung cancer (NSCLC).

In one embodiment, there is provided a method of reducing the growth or proliferation of a tumor, the method comprising contacting the tumor with double negative T cells (DNTs) and a PD-1 or PD-L1 inhibitor. Also provided is the use of DNTs and a PD-1 or PD-L1 inhibitor for reducing the growth or proliferation of a tumor. In one embodiment, the tumor is in vivo or in vitro. In one embodiment, the tumor is a solid tumor. In one embodiment, the tumor is in a subject with cancer and the method comprises administering the DNTs and the PD-1 or PD-L1 inhibitor to the subject.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIG. 1. DNTs inhibit tumor growth in xenograft models. Mice bearing A) H460 or B) A549 xenografts were treated i.v. with PBS or DNTs (10⁷/injection) for 2 or 3 times in the present of IL-2. Mice were sacrificed on day 24 (n=5/group), tumor volumes were calculated. Arrows indicate the days of PBS or DNTs injection. 2 injections of DNTs contained only 1^{st} and 2^{nd} DNT injections. Differences were calculated using two-way ANOVA followed by Bonferroni's post hoc test. *P<0.05 and ***P<0.001 compared to H460+PBS+IL-2 group in a, and A549+PBS+IL-2 group in b. Data shown are representative of three independent experiments.

FIG. 2. Cytotoxic molecules on DNTs. A) Ex vivo expanded DNTs were stained with indicated antibody (solid line) or isotype control antibody (filled histogram). B) Relative MFI was calculated compared to DNTs stained with isotype control antibody. Data from 5 donors are shown. C) DNT supernatants were collected, IFNγ, sTRAIL and TNFα were measured by ELISA. Each dot represents the data obtained from one healthy donor.

FIG. 3. DNTs upregulate PD-1 upon expansion. A) Expression of PD-1 on DNTs before and after expansion. B), Expression of PD-1 across various donors during expansion, result of six independent donors are shown. C) PD-1 expression of expanded DNTs after 48 hrs co-culture with lung cancer cell lines. **P<0.01, ***P<0.001, by unpaired t test.

FIG. 4. Anti-PD-1 increase DNT-mediated anti-tumor activity in vivo. H460 subcutaneously inoculated into the right flank of NSG mice and were allowed to reach ˜100 mm3. Tumor bearing mice were treated with saline or anti-PD-1 antibody Nivolumab 10 mg/kg ip injections starting 1 day before 1st DNTs infusion and repeated every 5 days. a, 2×10\hat{ }7 DNTs were injected i.v. either alone or into mice receiving Nivolumab. B), 2×10\hat{ }7 DNTs were injected s.c. alone or into mice receiving Nivolumab. Tumor volume as mean and SEM are shown. *p<0.05,**p<0.005,***p<0.001 by unpaired t test.

FIG. 5. Anti-PD-1 treatment increases DNT infiltration and tumor necrosis at tumor site. Representative histological images of A) CD3 stained sections and B) matched H&E and quantification of various treatment groups. Results are shown as mean and SEM. *p<0.5,**p<0.01,***p<0.001 by unpaired t test.

FIG. 6. Marker expression of tumor infiltrating DNTs from various treatment groups. DNTs 14 days post last infusion were analyzed from single cell suspension of resected tumor for A) surface markers or B), cytokines and C) intracellular markers. Results are shown as mean and SEM from 3 mice. *p<0.5, by unpaired t test.

FIG. 7. Ex vivo expanded DNT cells inhibit late stage tumor growth in xenograft models. NSG mice were inoculated subcutaneously with NCI-H460 (A, B, D) or XDC137 (C and E) in 50% Matrigel solution and grown to ˜100 mm3. After tumors were established, tumor bearing mice were randomized into groups and treated with peritumoral injection of IL-2 with or without DNT cells on day 0, 3 and 6. (A and C) Tumor volume was measured at indicated time points. Arrows indicate DNT cell treatments. Results represent one of three independent experiments, each consisting of 5 mice per treatment group (A), or one experiment consisting of 3 mice per treatment group (C). B. Survival of mice receiving IL-2 (control) or IL-2+DNT cells (DNT). D and E. Immunohistochemistry staining with anti-human CD3 antibody on resected tumor xenografts. Representative sections of CD3+ DNT cells within tumor xenograft from both groups are shown at 21 days for NCI-H460 xenografts (D) and at 71 days for XDC137 xenografts (E) Quantified CD3+ staining density of whole xenograft sections, as determined by digital analysis of positive stain per area analyzed. Each dot represents one mouse and horizontal bars represent the mean±SEM. Data shown are representative of 2 separate experiments. *p<0.05, **p<0.01, ***p<0.001, by two-tailed unpaired t-test (A, C, D and E) or log-rank (B)

FIG. 8. DNT cells upregulate PD-1 during interaction with NSCLC. Flow cytometric analysis of PD-1 expression on T cells from resected tissue compartment of lung cancer patients' cancer (CA), adjacent (ADJ), or normal lung tissue (NOR) (n=10). A. Frequencies of PD-1+ T cell subsets in patient lung tissue. B. Comparison of tumor infiltrating PD-1+ T cells subsets in cancer tissue. Each symbol represents an individual patient, bars represent mean value. *p<0.05, **p<0.01, ***p<0.001 by one-way ANOVA. C. Time course of PD-1 expression on expanded CD4, CD8 and DNT cells, results shown as mean±SEM expanded from 3 different donors. *p<0.05, **p<0.01, ***p<0.001, by two-tailed unpaired t-test. D. The expression of PD-L1 on NSCLC cell lines. NSCLC cell lines were stained with either anti-human PD-L1 (blue histograms) or control (red histograms), numbers represent MFI. E. PD-1 expression on DNT cells cultured alone or with various NSCLC cell lines for 48 hours. One representative result of two independent experiments is shown. F. Time course of PD-1 expression on expanded DNT cells, cultured alone or with various NSCLC cell lines for varying time points. Results represent the data obtained from 2 different donors.

FIG. 9. Intracellular cytokine analysis of DNT cells, either cultured alone or with various NSCLC cell lines. Expanded DNT cells were culture alone or co-cultured with indicated NSCLC cell lines for 48 hr and prior to analysis of indicated intracellular cytokines. % INF-γ and TNF-α positive DNT cells are shown.

FIG. 10. Treatment with anti-PD-1 alone has no effect on NCI-H460 xenograft growth or mouse survival. A. NSG mice were inoculated subcutaneously with NCI-H460 in 50% Matrigel solution and grown to ˜100 mm3, and treated with 10 mg/kg anti-PD-1 or PBS i.p. every 5 days to the end of the experiment. Tumor volume and recipient survival were monitored (n=5 for each group).

FIG. 11. Anti-PD-1 antibody enhances the efficacy of DNT cell-mediated inhibition of late-stage tumor growth. NSG mice were inoculated subcutaneously with NCI-H460 in 50% Matrigel solution and grown to ˜100 mm3. After tumors were established, tumor bearing mice were randomized and received peritumoral injection of DNT cells and IL-2 on day 0, 3 and 6, without or with anti-PD-1 antibody (10 mg/kg repeated every 5 days i.p., starting one day prior to 1st DNT cell infusion). A. Schematic diagram of the treatment protocol of NCI-H460 xenograft model. B. Survival of the mice treated with PBS control or DNT cells with or without anti-PD-1 (n=8 for each group). C. Anti-PD-1 treatment alone does not alter tumor growth. H460 subcutaneously inoculated into the right flank of NSG mice and were allowed to reach ˜100 mm3. Tumor bearing mice were treated with saline or anti-PD-1 antibody Nivolumab 10 mg/kg ip injections starting 1 day before 1st DNTs infusion and repeated every 5 days. 2×10\hat{ }7 DNTs were injected s.c. alone or into mice receiving Nivolumab. % tumor growth after DNT cell injection was determined. *p<0.05, **p<0.01, ***p<0.001, by log-rank test (C).

FIG. 12. Anti-PD-1 antibody enhances efficacy of DNT cell mediated anti-tumor response and DNT cell tumor infiltration in late stage tumor xenograft model. NSG mice were inoculated subcutaneously with NCI-H460 in 50% Matrigel solution and tumors were allowed to grow to ˜100 mm3. After tumors were established, tumor bearing mice were randomized and received intravenous injection of IL-2 (control treatment) or DNT cell plus IL-2 on days 0, 3 and 6. Additionally, some mice received PBS or anti-PD-1 antibody (10 mg/kg repeated every 5 days i.p., starting one day prior to 1st DNT cell infusion to the end of the experiment). A. Schematic diagram of the treatment protocol of NCI-H460 xenograft model. B. Humane end point survival of treated mice (n=10 for each group). C. Representative H&E staining of xenografts from indicated treatment groups 9 days post DNT cell infusion and percent necrotic area in tumors from indicated treatment groups calculated by histological analysis.

FIG. 13. Anti-PD-1 antibody enhances infiltration of cytotoxic DNT cells into tumor xenografts. Tumor-bearing NSG mice received peritumoral (A and B) or intravenous (C) injection of DNT cells with or without anti-PD1 treatment. A. Representative flow cytometric analysis of DNT cells pre-infusion and tumor infiltrating DNT cells 21 days post infusion. The data shown represent results from 2 independent experiments. B-C. Immunohistochemistry analysis of tumor infiltrating DNT cells. Nine days post DNT cell infusion, tumor xenografts were harvested and stained with anti-human CD3 antibody and quantified by Aperio Image-scope. Representative staining and analysis of tumor infiltrating DNT cells in indicated treatment groups are shown. Each dot represents one mouse and horizontal bars represent the mean±SEM. Data shown are representative of 2 separate experiments. **p<0.01, ***p<0.001, by one-way ANOVA.

FIG. 14 Flow cytometry analysis of tumor infiltrating DNT cells. Frequency of NKG2D+ or DNAM-1+ DNT cells (A). IFNγ+ and TNFα+ DNT cells (B), perforin+, granzyme B+ and CD107a+ DNT cells (C). Representative results shown as mean±SEM from 3 tumors of 2 separate experiments are shown. (*p<0.05 by two-tailed unpaired t-test).

### DETAILED DESCRIPTION

The advent of novel immunotherapies has revolutionized the treatment of cancer. However, the application of checkpoint inhibitors and vaccines show limitations as many patients still show incomplete responses and adverse events. Here, an adoptive T cell therapy that utilizes a rare population of CD4 and CD8 double negative T cells (DNTs) to target cancer is described.

Remarkably, as shown in the Examples, the use of a PD-1 inhibitor has been shown to increase DNT mediated anti-tumor activity in vivo. The combination of DNTs and PD-1 and/or PD-1L inhibitors are therefore expected to be useful for the treatment of subjects with cancer.

In one embodiment, there is provided a composition comprising double negative T cells (DNTs) and a PD-1 or PD-L1 inhibitor. Also provided is a kit comprising DNTs and a PD-1 or PD-L1 inhibitor, optionally in separate containers. In one embodiment, the kit further comprises instructions for treating cancer in a subject in need thereof.

PD-1 and PD-L1 inhibitors act to inhibit the association of the programmed death-ligand 1 (PD-L1) with its receptor, programmed cell death protein 1 (PD-1). Various PD-1 and/or PD-L1 inhibitors are known in the art. For example, in one embodiment the PD-1 or PD-L1 inhibitor is an antibody. In one embodiment, the PD-1 or PD-L1 inhibitor is an antibody or a fragment of antibody such as Fab, F(ab)², or single-chain variable fragment (scFv); or a nanobody, that selectively binds to PD-1 or PD-L1 and prevents the association of PD-L1 with PD-1. In one embodiment, the PD-1 inhibitor is Nivolumab. In another embodiment, the PD-1 inhibitor is Pembrolizumab or Cemiplimab. In one embodiment, the PD-1 inhibitor is Tislelizumab, Spartalizumab, Sintilimab, Toripalimab, Camrelizumab, Dostarlimab, MGA012 or AB122. In one embodiment, the PD-L1 inhibitor is selected from the group consisting of Atezolizumab, Avelumab and Durvalumab. In one embodiment, the PD-L1 inhibitor is Cosibelimab, Envafolimab or KN035.

As used herein, the term “double negative T cells” or “DNTs” refers to T cells that are CD4−, CD8−, and CD3+, as well as γδ-TCR+ and/or αβ-TCR+. DNTs may be obtained by a person of skill in the art. DNTs can be obtained by enriching using CD4 and CD8− depetion antibody cocktails. Optionally, in one embodiment the DNTs are α-GalCer-CD1d tetramer− and CTLA4−. In one embodiment, the DNTs express CD3-TCR complex and do not express CD4 and CD8. In one embodiment, the DNTs have the phenotype CD3+, γδ-TCR+ and/or αβ-TCR+, CD4−, CD8−. In one embodiment, the DNTs have the phenotype CD3+, γδ-TCR+ and/or αβ-TCR+, CD4−, CD8−, α-GalCer-CD1d tetramer-CD1d tetramer−, CTLA4−, CD25+, and CD44+. In one embodiment, the DNTs have the phenotype CD3+, CD4−, CD8−, α-GalCer-CD1d tetramer−, CTLA4−, CD44+. Optionally, the DNTs have the phenotype CD3+, CD4−, CD8−, α-GalCer-CD1d tetramer−, Jα24−, Vα14−, CD44+, CTLA4−. As used herein, the term “DNTs” includes DNTs that have been modified to express an exogenous protein such as a Chimeric Antigen Receptor (CAR).

In one embodiment, the DNTs are recombinant cells that have been modified to express one or more exogenous proteins. For example, in one embodiment, the DNTs described herein express a receptor with a high avidity to a cancer biomarker, such as a protein expressed on the surface of a cancer cell. In one embodiment, the DNTs express a Chimeric Antigen Receptor (CAR) that preferentially binds to a cancer cell. For example, in one embodiment the DNTs described herein express one or more receptors that bind to CD33, CD19, CD20, CD123 and/or LeY.

In one embodiment, the DNTs may be obtained from a sample comprising peripheral blood mononuclear cells (PBMC). In one embodiment, the sample is a blood sample. In one embodiment, the sample is an apheresis sample, or an enriched leukapheresis product such as a leukopak. In one embodiment, the sample is a bone marrow, spleen or lymph node sample. Optionally, the sample is from one or more healthy donors. In one embodiment, the sample is from a subject with cancer or suspected of having cancer and the DNTs are used to treat the subject with cancer.

In one embodiment, the DNTs are expanded in vitro or ex vivo before their administration or use for the treatment of cancer as described herein. Exemplary methods for isolating and expanding DNTs are described in U.S. Pat. No. 6,953,576 “Method of Modulating Tumor Immunity”, PCT Publication No. WO2007/056854 “Method of Expanding Double Negative T Cells”, and PCT Publication No. WO2016/023134 “Immunotherapy for the Treatment of Cancer” all of which are hereby incorporated by reference in their entirety.

In one embodiment, the DNTs are autologous DNTs obtained from a subject, such as a subject with cancer or suspected of having cancer. In another embodiment, the DNTs are allogenic, such as DNTs obtained from one or more subjects without cancer. In one embodiment, the DNTs are obtained from one or more healthy donors.

The DNTs and PD-1 or PD-L1 inhibitors may be combined in a composition, optionally with a pharmaceutically acceptable carrier as described herein.

In one embodiment, there is also provided a kit comprising DNTs and a PD-1 or PD-L1 inhibitor. In one embodiment, the kit further comprises instructions for performing a method as described herein, such as for the treatment of cancer or for reducing the growth or proliferation of a tumor. In one embodiment, the DNTs and the PD-1 or PD-L1 inhibitor are in separate containers. In one embodiment, the DNTs and the PD-1 or PD-L1 inhibitor are in the same container, optionally as a composition with a pharmaceutically acceptable carrier.

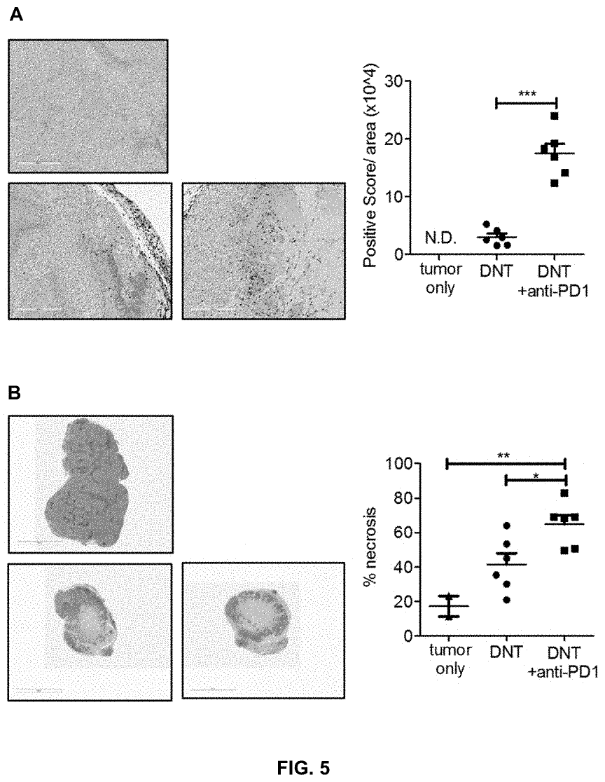

The combination of DNTs and PD-1 or PD-L1 inhibitors as described herein exhibit a number of characteristics that render them particularly useful for the treatment of cancer. For example, in one embodiment the PD-1 or PD-L1 inhibitor increases DNT-mediated anti-tumor activity. In one embodiment, the PD-1 or PD-L1 inhibitor increases DNT tumor infiltration. In one embodiment, the PD-1 or PD-L1 inhibitor increases DNT-mediated cytotoxicity. As shown in the Examples the combination of DNTs and PD-1 blockade resulted in a significant reduction in tumor growth relative to treatment with DNTs alone, while no tumor reduction was observed for PD-1 blockade alone. Furthermore, compared to DNT cell transfer alone, the use of DNT cells with PD-1 blockade led to a greater infiltration of T cells with high expression of cytotoxicity markers. DNTs were previously known to express low levels of PD-1, and it was not known whether the combined use of DNTs and a PD-1 or PD-L1 inhibitor would be useful for the treatment of cancer or for reducing the growth or proliferation of a tumor. Here, the combined use of DNTs and PD-1 blockade has been demonstrated to have a synergistic effect for reducing tumor growth in vivo and improve survival times in a xenograft mouse model of lung cancer. PD-1 blockade such as by the use of a PD-1 or PD-L1 inhibitor is known to exert anti-tumor activity by reinvigorating exhausted T cells, but as shown in the Examples, the combined use of DNTs and PD-1 blockade surprisingly has also been demonstrated to increase the tumor infiltration of DNT cells. In addition, as shown in FIG. 5, the use of PD-1 inhibitor and DNTs led to a dramatic increase in tumor necrosis relative to the use of DNTs alone. Also, high PD-1 expression in tumor infiltrating DNT cells was surprisingly observed in human lung cancer samples as well as the induction of PD-1 in DNTs by tumor cells

In one embodiment, there is provided is a method of treating cancer in a subject in need thereof. In one embodiment, the method comprises administering to the subject an effective amount of DNTs and a PD-1 or PD-L1 inhibitor. Also provided is the use of an effective amount of DNTs and a PD-1 or PD-L1 inhibitor for the treatment of cancer in a subject in need thereof.

As used herein, the term “cancer” refers to one of a group of diseases caused by the uncontrolled, abnormal growth of cells that can spread to adjoining tissues or other parts of the body. In one embodiment, the cancer is a lung cancer such as NSCLC. In one embodiment, the cancer is a melanoma. In one embodiment, the cancer is pancreatic cancer. In one embodiment, the cancer is a hematological malignancy such as multiple myeloma, leukemia or lymphoma.

The term “cancer cell” refers to a cell characterized by uncontrolled, abnormal growth and the ability to invade another tissue or a cell derived from such a cell. Cancer cells include, for example, a primary cancer cell obtained from a patient with cancer or cell line derived from such a cell. In one embodiment, the cancer cell is a lung cancer cell such as a NSCLC cell. In one embodiment, the cancer cell is a melanoma cell. In one embodiment, the cancer cell is a pancreatic cancer cell. In one embodiment, the cancer cell is a hematological cancer cell such as a leukemic cell.

The term “tumor” refers to a collection of cancer cells. In one embodiment, the tumor is a lung cancer tumor such as a NSCLC cell. In one embodiment, the tumor is a pancreatic tumor. In one embodiment, the tumor is a solid tumor.

The term “subject” as used herein includes all members of the animal kingdom including mammals, and suitably refers to humans. Optionally, the term “subject” includes mammals that have been diagnosed with cancer or are in remission. In one embodiment, the term “subject” refers to a human having, or suspecting of having, melanoma or lung cancer such as NSCLC. In one embodiment, the term “subject” refers to a human having, or suspecting of having, pancreatic cancer or a hematological cancer.

In one embodiment, the methods and uses described herein involve the administration or use of an effective amount of DNTs and a PD-1 or PD-L1 inhibitor, such as by injection. In one embodiment, the methods and uses described herein involve the administration or use of a plurality of doses of DNTs. In one embodiment, the methods and uses described herein involve the administration or use of a plurality of doses of the PD-1 or PD-L1 inhibitor.

As used herein, the phrase “effective amount” or “therapeutically effective amount” means an amount effective, at dosages and for periods of time necessary to achieve the desired result. For example, in the context or treating cancer, an effective amount is an amount that for example induces remission, reduces tumor burden, and/or prevents tumor spread or growth of cancer cells compared to the response obtained without treatment. In one embodiment, an effective amount of the PD-1 or PD-L1 inhibitor is an amount that increases DNT-mediated anti-tumor activity, increases DNT tumor infiltration and/or increases DNT-mediated cytotoxicity.

Effective amounts may vary according to factors such as the disease state, age, sex and weight of the animal. The amount of a given dosage that will correspond to such an amount will vary depending upon various factors, such as the pharmaceutical formulation, the route of administration, the type of disease or disorder, the identity of the subject or host being treated, and the like, but can nevertheless be routinely determined by one skilled in the art. In one embodiment, the DNTs and PD-1 or PD-L1 inhibitor are administered to a subject by injection. In one embodiment, the injection is an intravenous injection. In one embodiment, the injection for accessible tumors (cutaneous, subcutaneous, or superficial) and/or palpable tumors is a subcutaneous injection around the tumor or an intratumoral injection at or around the tumor site.

In one embodiment, the combination of the PD-1 or PD-L1 inhibitor and DNTs may be used to reduce the growth or proliferation of cancer cells in vitro, ex vivo or in vivo. As used herein, “reducing the growth or proliferation of a cancer cell” refers to a reduction in the number of cells that arise from a cancer cell as a result of cell growth or cell division and includes cell death. The term “cell death” as used herein includes all forms of killing a cell including cell lysis, necrosis and/or apoptosis. In one embodiment, the combination of DNTs and a PD-1 or PD-L1 inhibitor may be used to kill cancer cells in vitro, ex vivo or in vivo.

In one embodiment, the DNTs and PD-1 or PD-L1 inhibitor may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The term “pharmaceutically acceptable” means compatible with the treatment of animals, in particular, humans.

In one embodiment, the DNTs and PD-1 or PD-L1 inhibitor are administered to the subject at the same time, optionally as a composition comprising the DNTs and the PD-1 or PD-L1 inhibitor, or as two separate doses. In one embodiment, the DNTs and PD-1 or PD-L1 inhibitor are used or administered to the subject at different times. For example, in one embodiment, the DNTs are for use or administered prior to, or after administering a PD-1 or PD-L1 inhibitor. In one embodiment, the DNTs are for use or administered prior to, or after the PD-1 or PD-L1 inhibitor separated by a time of no more than about 90 days, 60 days or 45 days. In one embodiment, the DNTs are for use or administered prior to, or after the PD-1 or PD-L1 inhibitor separated by a time of no more than about 45 days, 40 days, 35 days, 30 days, 25 days, 20 days, 15 days, 2 weeks, 7 days, 6 days, 5 days, 4 days or 3 days.

In one embodiment, the DNTs are for use or administered prior to, or after the PD-1 or PD-L1 inhibitor separated by a time of about 12, 24 or 36 hours. In one embodiment, the DNTs are for use or administered prior to, or after the PD-1 or PD-L1 inhibitor separated by a time of about 1 minute, 2 minutes, 5 minutes, 10 minutes, 30 minutes, 45 minutes, 1 hour, 1.5 hours, 2 hours, 3 hours, 4 hours, 5 hours, 8 hours, 10 hours, 12 hours 16 hours, or 24 hours. In one embodiment, the DNTs are for use or administered prior to, or after the PD-1 or PD-L1 inhibitor separated by a time of between about 1 hour and 48 hours, optionally between about 12 hours and 36 hours. In one embodiment, the DNTs are for use or administered prior to, or after the PD-1 or PD-L1 inhibitor separated by a time of no more than between about 1 hour and 60 days. In one embodiment, the DNTs are for use or administered prior to, or after, the PD-1 or PD-L1 inhibitor separated by a time of no more than between about 1 day and 60 days, optionally no more than between about 2 days and 45 days, between about 2 days and 30 days, between about 2 days and 14 days or no more than about 7 days. In one embodiment, the DNTs are for use or administered to the subject the same day as the PD-1 or PD-L1 inhibitor.

In one embodiment, the DNTs are for use or administered prior to, or after, the PD-1 or PD-L1 inhibitor separated by a time such that both the DNTs and the PD-1 or PD-L1 inhibitor are present in the subject at the same time. For example, in one embodiment the DNTs and the PD-1 or PD-L1 inhibitor are present in the subject at the same time such that the PD-1 or PD-L1 inhibitor increases DNT-mediated anti-tumor activity, DNT tumor infiltration and/or DNT-mediated cytotoxicity.

In one embodiment, the DNTs are for administration, use or formulated for use parenterally. For example, in one embodiment the DNTs are for administration, use or formulated for intravenous (IV), subcutaneous (SC) or intradermal (ID) use or administration. In one embodiment, the DNTs are for administration, use or formulated for use within or at a tumor site. For example, in one embodiment the DNTs are for intratumoral or peritumoral use or administration.

In one embodiment, the PD-1 or PD-L1 inhibitor is for administration, use, or formulated for use parenterally. For example, in one embodiment, the PD-1 or PD-L1 inhibitor is for administration, use or formulated for intravenous (IV), subcutaneous (SC) or intradermal (ID) administration. In one embodiment, the PD-1 or PD-L1 inhibitor is for administration, use or formulated for use within or at a tumor site. In one embodiment, the PD-1 or PD-L1 inhibitor is for intratumoral or peritumoral use or administration.

Optionally, the DNTs and the PD1 or PD-L1 inhibitor are for use or administration to the subject using the same route of administration or different routes of administration. For example, in one embodiment, the method comprises intravenous use or administration of the PD-1 or PD-L1 inhibitor and subcutaneous use or administration of the DNTs to a tumor site.

In one embodiment, the method described herein comprises administering to the subject a plurality of separate doses of DNTs. In one embodiment, the method described herein comprises administering to the subject a plurality of separate doses of the PD-1 or PD-L1 inhibitor.

In one embodiment, one dose of DNT may be administrated or used for the treatment of cancer in a subject in need thereof, optionally in combination with one or more doses of a PD-1 or PD-L1 inhibitor. For example in one embodiment only a single dose of DNTs is administrated or used and one or more doses of the PD-1 or PD-L1 inhibitor are administered or used for the treatment of cancer within a set period. In one embodiment, a single dose of DNTs is administered or used within a set period while a plurality of doses of a PD-1 or PD-L1 inhibitor are administered or used within the set period. The set period may be is 3 years, 2 years, 18 months, 1 year or 6 months.

In one embodiment two or more separate doses of DNTs may be administered or used for the treatment of cancer in a subject in need thereof. In one embodiment, the methods and uses described herein include a first dose of DNTs and at least one additional dose of DNTs. In one embodiment, at least one additional dose is for use or administration between 1 day and 6 months after the first dose of DNTs. In one embodiment, the at least one additional dose is for use or administration within 3 days, 5 days, 1 week, 10 days, 2 weeks, 3 weeks, 4 weeks, 5 weeks, 6 weeks, 7 weeks, 8 weeks, 12 weeks, 16 weeks, 20 weeks, 24 weeks or 6 months after the first dose of DNTs. In one embodiment, the at least one additional dose is for use or administration within between 1 day and 12 weeks after the first dose of DNTs, between 1 day and 8 weeks after the first dose of DNTs, between 1 days and 4 weeks after the first dose of DNTs or between about 3 days and 10 days after the first dose of DNTs.

In one embodiment, the method comprises the use or administration of three separate doses of DNTs to the subject within a predetermined time-period. For example, in one embodiment, the method comprises the use or administration of three separate doses of DNTs to the subject within 10 days, 2 weeks, 3 weeks, 4 weeks, 5 weeks, 6, weeks, 7 weeks 8 weeks, 12 weeks, 16 weeks, 20 weeks, 24 weeks or 1 year. Optionally, the three separate doses are for use or administration to the subject separated by a time period of at least 3 days, 5 days, 7 days, 10 days, 2 weeks, 3 weeks, 4 weeks, 5 weeks or 6 weeks.

In one embodiment, the method further comprises the use or administration of a plurality of separate doses of the PD-1 or PD-L1 inhibitor. For example, in one embodiment two or more separate doses of the PD-1 or PD-L1 inhibitor may be administered or used for the treatment of cancer in a subject in need thereof in combination with the administration or use of DNTs. In one embodiment, the methods and uses described herein include a first dose of a PD-1 or PD-L1 inhibitor and at least one additional dose of a PD-1 or PD-L1 inhibitor. In one embodiment, the at least one additional dose is for use or administration between 1 day and 6 weeks after the first dose of PD-1 or PD-L1 inhibitor. In one embodiment, the at least one additional dose is for use or administration within 3 days, 5 days, 1 week, 10 days, 2 weeks, 3 weeks, 4 weeks, 5 weeks or 6 weeks after the first dose of the PD-1 or PD-L1 inhibitor. In one embodiment, the at least one additional dose is for use or administration within between 2 days and 3 weeks after the first dose of PD-1 or PD-L1 inhibitor, or between about 3 days and 10 days after the first dose of the PD-1 or PD-L1 inhibitor.

In one embodiment, the methods described herein comprise the administration or use of two or more doses of DNTs and two or more doses of a PD-1 or PD-L1 inhibitor. In one embodiment, at least one of the two or more doses of the PD-1 or PD-L1 inhibitor are for administration or use within 2 days, 3 days, 4 days, 5 days, 6 days 1 week, 10 days, 2 weeks, 3 weeks, or 4 weeks of the administration or use of at least one of the doses of DNTs. In one embodiment, at least one of the two or more doses of the PD-1 or PD-L1 inhibitor are for administration or use the same day as the administration or use of the DNTs.

The following non-limiting examples are illustrative of the present disclosure:

### Example 1: Combination of DNT Cells with Checkpoint Blockade Therapy Increases DNT Effectiveness

### Materials and Methods

### Antibodies

Antibodies specific for CD3 (clone HIT3a), CD4 (clone OKT4), CD8 (clone HIT8a), CD69 (clone FN50), CD25 (clone PC61), NKG2D (clone 1D11), DNAM-1 (clone 118A), Fas ligand (FasL) (clone NOK-1), NKp30 (clone P30-15), NKp44 (clone P44-8), NKp46 (clone 9E2), perforin (clone B-D14), granzyme B (clone GB11), anti-human NKG2D (clone 1D11), anti-human DNAM-1 (clone 11A8), anti-human NKp30 (clone P30-15), anti-human FasL (clone NOK-1), anti-human NKp44 (clone P44-8), anti-human PD-1, anti-human LAG3, anti-human TIM3, as well as isotype antibodies mouse IgG1, κ (clone RMG1-1), mouse IgG2α, κ (clone RMG2a-62), mouse IgG2β, κ (clone 27-35) and rat IgG1, γ (clone G0114F7) were purchased from Biolegend.

### Cell Culture

DNTs were expanded ex vivo from healthy donors as described previously (1). In brief, blood samples were obtained from healthy donors and DNTs were enriched by depleting CD4+ and CD8+ cells using CD4− and CD8− depletion antibody cocktails (Stemcell Technologies). After incubating at room temperature for 20 mins, blood was spun down at 1200 g for 20 mins. Cell suspension was poured into one new Falcon tube and washed twice with 2% FBS containing PBS. Therefore, DNTs were enriched. The enriched DNTs were cultured in 24-well plates pre-coated with 5 μg/ml anti-CD3 antibody (OKT3, eBioscience) for 3 days in RPMI-1640 (Thermo Fisher Scientific) supplemented with 10% FBS (Sigma) and 250 IU/ml IL-2 (Proleukin). DNTs were restimulated with 0.01-1 μg/ml soluble anti-CD3 and IL-2 every 2-3 days. DNTs were harvested on day 14-20 and the purity was assessed by flow cytometry prior to experiments. The mean purity of DNTs used in the study was ˜94%.

The NSCLC cell lines H2279, H460, H125 and A549 were obtained from ATCC. Primary NSCLC cell lines 12, 178, 426, 277, 655, 229, 239 and 137 were derived from NSCLC PDX models (Table 2), which were established using a protocol approved by the University Health Network (UHN) Research Ethics Board. Briefly, primary lines were established from single cell suspensions of their corresponding PDX grown in immune deficient mice. Mutation information of primary NSCLC cell lines was profiled by OncoCarta Panel v1.0 (Agena Bioscience, San Diego, Calif.). All cell lines were maintained in DMEM/F12 (Gibco) supplemented with 10% FBS and used at less than 15 passages in vitro.

### Cytotoxicity and Blocking Assays

NSCLC cell lines were labelled at 1×10⁶ cells/ml with 5 μM florescent Vybrant™ DiO in PBS (ThermoFisher Scientific) for 15 mins at 37° C. After two washes the DiO-labelled targets were added to 96-well flat-bottom microtiter plates in 100 μl DMEM/F12 with 10% FBS at 1×10⁵ cells/ml. DNTs were added at different E:T ratios. To maintain the survival of DNTs, IL-2 was added to the medium. Plates were incubated in a humidified atmosphere of 5% CO2 and 37° C. In some cases, only 250 IU/ml IL-2 or supernatants from DNTs stimulated with 250 IU/ml IL-2 were added. After 14 hrs, non-adherent cells were collected and transferred to a new microtiter plate. Remaining adherent cells were dissociated with 0.25% trypsin-EDTA solution (Sigma) and collected with non-adherent wells. To stain for dead cells, both cell fractions were resuspended together in a solution containing 3 μM TO-PRO-3 (ThermoFisher Scientific) and analyzed by flow cytometry to determine the frequency of DiO+TO-PRO-3+ target cells. The specific cytotoxicity of DNTs against NSCLC cell was calculated by the formula % Specific Killing=100%×(% DiO⁺TO-PRO-3⁺, in DNT:target containing wells−% DiO⁺TO-PRO-3⁺, in target alone wells)/(100−% DiO⁺TO-PRO-3⁺, in target alone wells). The E:T EC50 was calculated using a non-linear regression fit of all E:T ratios in Table 1.

For in vitro blocking assays, blocking antibodies or isotype matched controls were cultured with DNTs for 1 hr prior to co-incubation with target cells at E:T ratio=5:1 for 14 hr. Concanamycin A (CMA) was used to block perforin and granzyme B activity. Percent of cytotoxicity inhibition was calculated by measuring the change in cytotoxicity observed between co-cultures containing blocking antibody and isotype control, vehicle or CMA and media.

### Histology

FFPE tissue were generated from resected tumor harvested 14 days after the last DNT injection. 4 μm sections were stained for H&E or human CD3 at PMH AMPL. Sections were scanned and analyzed in Aperio Imagescope. Necrotic area as observed in H&E tissue were quantified by determining necrotic area/whole tumor area. CD3 infiltrating DNT are evaluate by positive signal density/whole tumor area.

### Xenograft Model

6-8 week-old male NOD.Cg-Prkdcscid II2rgtm1WjI/SzJ (NSG) mice were subcutaneously inoculated with H460 cells or A549 cells (1×10⁶/mouse) on day 0. Three days later, mice were treated i.v. with PBS or DNTs (2×10⁷/mouse) on days 3 and 7. For established tumor model, H460 cells were allowed to grow until average tumor volume reached ˜100-200 mm³ prior to DNT inoculation. Mice were given anti-PD-1 antibody (Nivolumab 10 mg/kg) i.p. starting 1 day before DNT infusion and repeated every 5 days. DNT were either infused i.v. or s.c. near the tumor on days 0, 3, and 7. Mice were sacrificed when the tumor diameter reached 1.5 cm. Tumor volume was calculated by length×width2×0.52.

### Tumor Infiltrating DNT Analysis

14 days after the last DNT infusion, tumor were resected, with half of tumor fixed for histological examination and half for single cell analysis. For single cells, tumor were digested in a solution of DNase I and collagenase D (DNase: 0.2 mg/ml; collagenase D: 1 mg/ml) at 37° C. in 5% CO2 for 30 min mix well per 10 min. After digestion, necrotic debris were removed using a Dead Cell removal Kit (Miltenyi). Isolated TILs were analyzed directly for surface expression of CD45 and additional surface makers or restimulated in the presence of PMA/ionomycin cocktrail (eBioscience) with protein transport inhibition (eBioscience) 4 hr prior to intracellular analysis. For CD107a analysis, anti-CD107a-APC were added to intracellular stimulation cocktail.

### Statistical Analysis

All graphs and statistical analyses were performed with GraphPad Prism 6. The data were analyzed by two-tailed Student's t-test, one-way ANOVA followed by Bonferroni's post hoc test and two-way ANOVA followed by Bonferroni's post hoc test. The results were expressed as mean±SD. Statistical significance was set as P<0.05.

### Results

### Ex Vivo Expanded DNTs Effectively Lyse Human Lung Cancer Cells In Vitro and Inhibit Tumor Growth in a Xenograft Model

Recently it was found that ex vivo expanded DNTs were cytotoxic towards human primary AML blasts and could reduce leukemia burden in PDX models of AML (1). To test the cytotoxic potential of DNTs against lung cancer, cells expanded from 8 healthy donors were cocultured with 8 primary and 4 established human lung small cell lung cancer (NSCLC) cell lines at varying E:T ratios (Table 1). Flow cytometry was used to measure cytotoxicity against NSCLC. Although cytotoxicity varied between different NSCLC lines, DNTs from all tested donors showed dose-dependent cytotoxicity towards both primary and established lung cancer cells (Table 1). The majority of NSCLC lines tested were highly susceptible to DNT-mediated lysis, with an E:T ratio EC50 of less than 10, such that an E:T ratio of 10:1 is capable of lysing 50% of NSCLC lines in cocultures. A549, and primary NSCLC lines 239, 137 were less susceptible, with a specific lysis E:T EC50 of greater than 16.

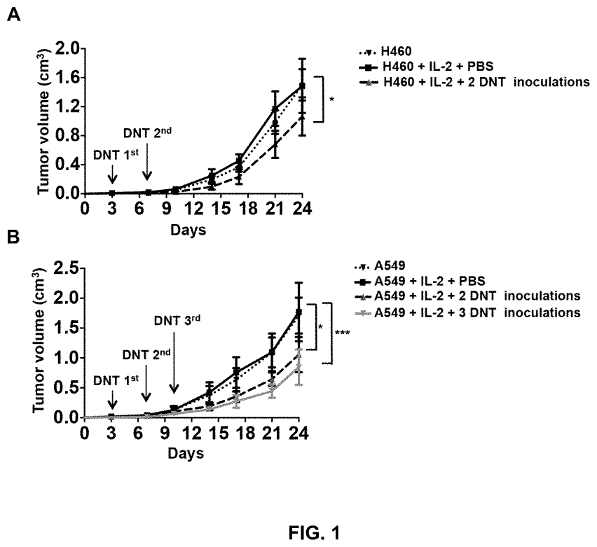

To further determine the anti-tumor effect of DNTs in vivo, H460 and A549 lung cancer cells were used to test the ability of DNTs to inhibit tumor growth in xenograft models (FIG. 1). NSG mice were subcutaneously injected with H460 or A549 cells followed by intravenous infusion of ex vivo expanded DNTs for 2 or 3 times post-tumor inoculation. To assist survival and proliferation of DNTs in mice, a low dose of IL-2 was administered i.p. twice a week. Neither H460 nor A549 tumor growth was remarkably affected by IL-2 treatment alone. However, a significant reduction in H460 tumor growth was observed between day 17 and day 24 in mice that received 2 injections of DNT treatments, with tumor volume being reduced by 34.26±17.81% on day 24 (FIG. 1a). Similarly, 2 and 3 DNT cell treatments resulted in 40.38%±14.83% and 51.05±7.29% reduction in A549 tumor volume, respectively on day 24 (FIG. 1b). Compared to 2 injections of DNTs, 3 injections of DNTs led to a greater inhibition of tumor growth, therefore, 3 injections of DNTs were given in the following experiments. These data demonstrate that adoptive transfer of DNTs after tumor inoculation can significantly inhibit lung cancer xenograft growth.

Expanded DNTs have a Cytotoxic Phenotype

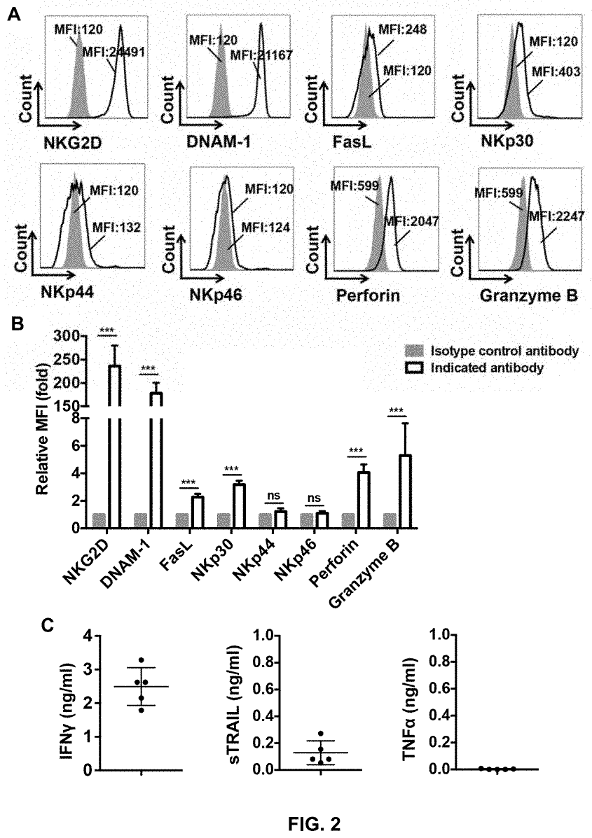

Immune mediated cytotoxicity is dependent on engagement of receptor-ligand interactions between effector and target cells (1, 10). To determine the mechanisms involved in DNT-mediated cytotoxicity toward lung cancer, we screened DNTs for expression of surface receptors known to be involved in immune cell mediated cytotoxicity, including the family of natural cytotoxicity receptors (NCR), NKp30, NKp44 and NKp46. Expanded DNTs showed a >150-fold increase in MFI values for NKG2D and DNAM-1, and a 2-fold increase in NKp30 and FasL expression compared to isotype controls (FIG. 2a, b). Expression of NKp44 and NKp46 was not detected. Expanded DNTs also expressed intracellular perforin and granzyme B (FIG. 2a, b) and secreted IFNγ and sTRAIL, but not TNFα (FIG. 2c). Collectively, surface expression analysis suggest DNT cells may be poised for cytotoxicity.

### DNT Cells Upregulate PD-1 During Expansion

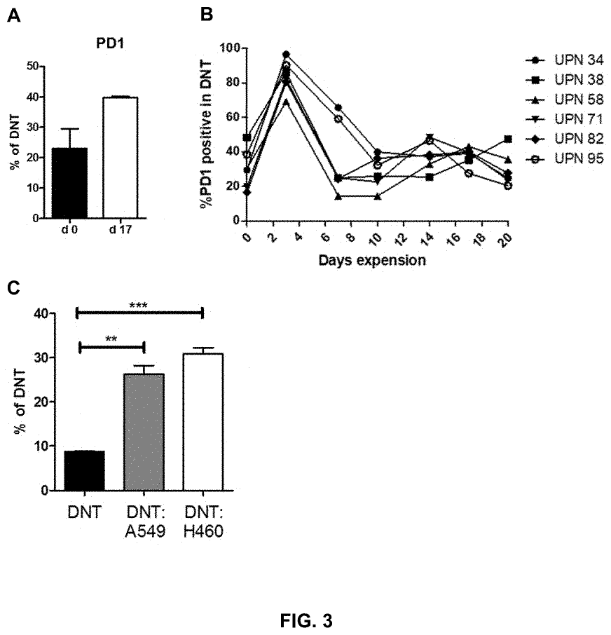

The TME exerts immune suppressive functions through PD-L1 and other factors. As such, the effectiveness of ACT is hindered due to the expression of PD-1 on the infusion products (8-9). To determine if PD-1 was also expressed on DNT cells, DNT was screened for PD-1 expression. Relative to unstimulated DNT cells, after expansion, a proportion of DNT cells upregulated PD-1 (FIG. 3a), but to a lower extent than cytotoxic markers (FIG. 2a). To determine the kinetics of PD-1 expression on DNT cells, the expression of PD-1 was followed on various days following DNT expansion. Though donor to donor variation was observed in PD-1 expression at day 0 (8%-48%), all donors transiently upregulated PD-1 expression upon expansion, with the greatest level of PD-1 expression at 3 days and returning to basal or slightly elevated levels by day 17. These observations suggested that PD-1 is dynamically expressed and regulated by activation or their environmental milieu. Consistent with this, it was found that PD-1 expression from PD-1low DNT donors upregulated PD-1 expression by 2.9-fold and 3.5-fold when coculture with A549 or H460 respectively, suggesting that lung cancer may exert additional regulatory functions on DNT, even in PD-1 low DNT donors.

### Anti-PD-1 Therapy Increase DNT-Mediated Anti-Tumor Activity In Vivo

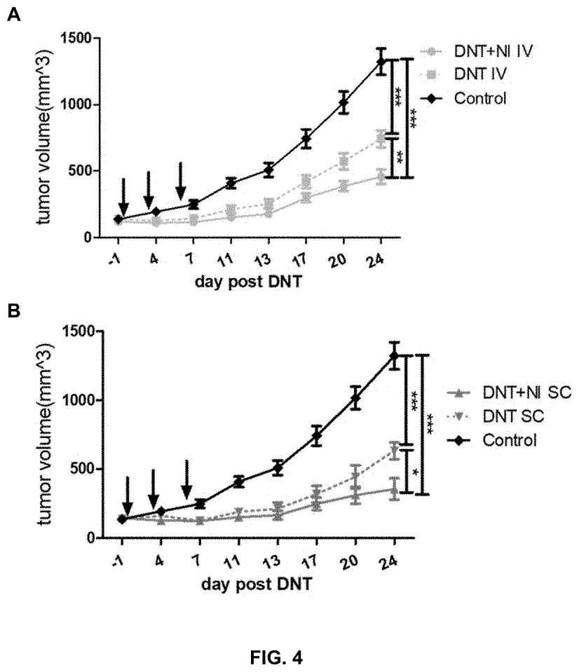

As DNT have the propensity to upregulate PD-1 during their expansion and in the presence of lung cancer, it was determined if addition of anti-PD-1 may augment DNT-mediated anti-tumor activity. To accomplish this, an established xenograft model of H460 was used, a cell line that expressed PD-L1 under natural regulatory promotors. H460 were allowed to grow until 100 mm³ size prior to inoculation of ex vivo expanded DNT cells. Additionally, mice were given anti-PD-1 to block the PD-1/PD-L1 pathway or control injection. Similar to our previous observations (FIG. 1), DNT treatment led to reduced tumor growth when infused intravenously (i.v) (FIG. 4a). Interestingly, when DNTs were injected subcutaneously (s.c.) near the tumor site, DNT cells also reduced tumor growth (FIG. 4b), suggesting that the route of injection does not alter DNT mediated anti-tumor activity.

Consistent with role of PD-1/PD-L1 pathway in mediating T cell suppression, addition of anti-PD-1 antagonist antibody, resulted in a further decrease of tumor volume irrespective of the route of DNT inoculation. For i.v. treatment, combination therapy resulted in a decrease of 60.3% versus 46.1% with DNT only (FIG. 4a). Similarly, when DNT are given s.c., combination therapy resulted in a decrease of 61.5% versus 50% when compared with DNT only (FIG. 4b). Taken together, combination therapy with anti-PD-1 antibody significantly augmented DNT cell-mediated tumor reduction over DNT treatment alone, indicating that DNT cells were responsive to immune checkpoint blockade therapy.

### PD-1 Blockade Increases Infiltration and Activation of DNT Cells

To determine how anti-PD-1 augmented DNT cell activity, we explored the number and phenotype of subcutaneously inoculated DNT cells in combination with anti-PD-1 blockade. Histological analysis revealed that tumor receiving combination treatment, had a 5.8-fold increase in DNT cells infiltrating the tumor bed relative to mice given DNT alone (FIG. 5a). The increase in DNT infiltration coincided with an increase in tumor necrosis as measured by hematoxylin and eosin (H&E) (FIG. 5b). Analysis of H&E stained tumor tissue shortly after DNT treatment revealed that anti-PD-1 significantly increased the proportion of necrotic area detected within tumors from mice receiving combination treatment (64.9±11.7% vs 41.3±14.5%; FIG. 5B-D). Overall, the histological differences we observed with combination of DNT and anti-PD-1 resulted in significantly increased DNT infiltration of tumors and reduced tumor growth.

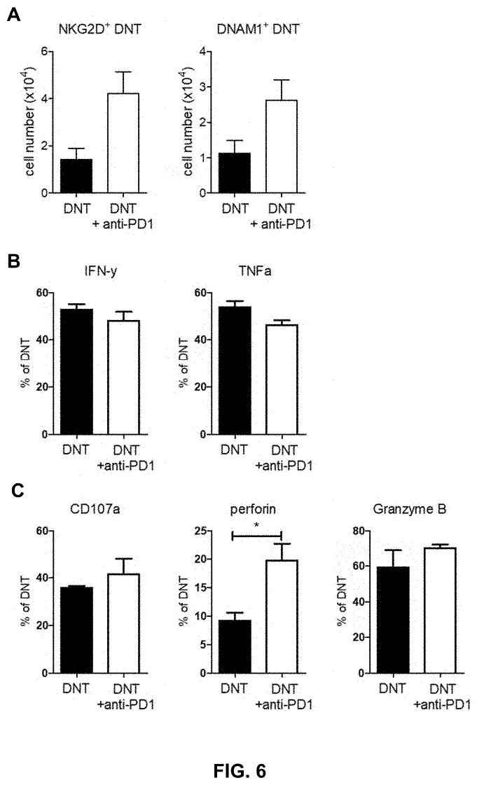

Consistent with its role in mediating T cell suppression, blockade of PD-1 increased tumor infiltrating DNT cells expressing high levels of cytotoxic markers and cytolytic granules. Though not reaching statistical differences, the number of tumor infiltrating DNTs expressing NKG2D and DNAM1 trended towards increased density in anti-PD-1 treated mice (FIG. 6a), whereas no difference was observed in DNT ability to secrete IFNg or TNFα (FIG. 6b). This lack of statistical difference is likely attributed to the already high expression of these markers on DNT upon expansion. Importantly, ex vivo restimulation of DNT from tumors of anti-PD-1 treated mice were found to have a significantly higher expression of perforin suggesting that blockade of anti-PD-1 led to increased infiltration of DNT cells capable of cytolytic granule secretion (FIG. 6c).

### Example 2: Investigations into the Use of PD-1 Inhibitors with DNTs for the Treatment of Cancer

Ex Vivo Expanded DNT Cells from Healthy Donors can Target Advanced Late-Stage Lung Cancer Xenografts

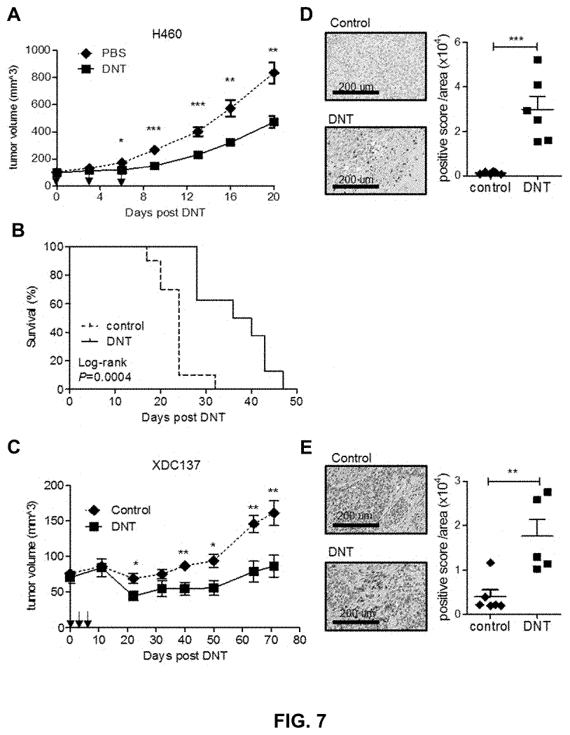

To determine whether DNT cells can target late-stage lung cancer in vivo, two late-stage xenograft models were generated. An NSCLC established cell line NCI-H460 and a patient-derived adenocarcinoma xenograft cell line XDC137 were inoculated subcutaneously (s.c.) into the flanks of sublethally irradiated NSG mice and let grown to ˜100 mm³. Tumor-bearing mice were then treated subcutaneously with 3 peritumoral injections of ex vivo expanded DNT cells or CD8 T cells in 3-4 days intervals. For the more aggressive NCI-H460 model, the PBS treated control tumor reached end-point by 20 days post treatment (FIG. 7a). However, DNT cell treatment resulted in a significant reduction of tumor growth as early as 6 days post 1^{st} DNT cell injection. At 20 days post DNT cell treatment NCI-H460 tumor volume was reduced by 43.3±15.9%, from 834.2±234.8 mm³ in the control group to 473.2±132.9 mm³ in the DNT cell treated group (FIG. 7A). Additionally, DNT cell-mediated inhibition of tumor growth led to a significant increase in the survival of NCI-H460 tumor-bearing mice, with a humane endpoint extending from median 24 days to 38 days (FIG. 7B). Though patient-derived xenograft model XDC137 grew much slower than NCI-H460, with humane endpoint not being reached by 71 days of observation, DNT cell treatment significantly reduced XDC137 xenograft volume from 160.8±39.5 mm³ in the PBS control group to 86.2±34.8 mm³ in the DNT cell treated group (FIG. 7C), resulting in a 46.4±21.6% reduction in tumor volume. These results show that adoptive transfer of healthy donor-derived DNT cells can significantly inhibit the growth of both aggressive and slow growing lung cancer xenografts.

As DNT cells were found in lung cancer patient tumor tissue (9), it was next determined whether DNT cells would be detectable within tumor xenografts at experimental endpoints. Using immunohistochemical staining for human CD3⁺ cells, DNT cells were detected infiltrating both aggressive xenograft, NCI-H460 (FIG. 7D) and slower growing xenograft, XDC137 (FIG. 7E), at days 21 and day 71, respectively.

### Tumor Infiltrating and Ex Vivo Expanded DNT Cells Express PD-1

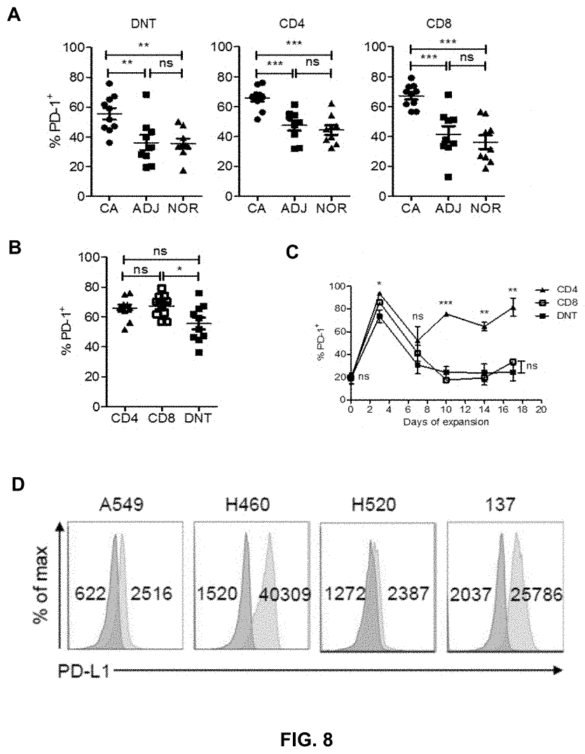

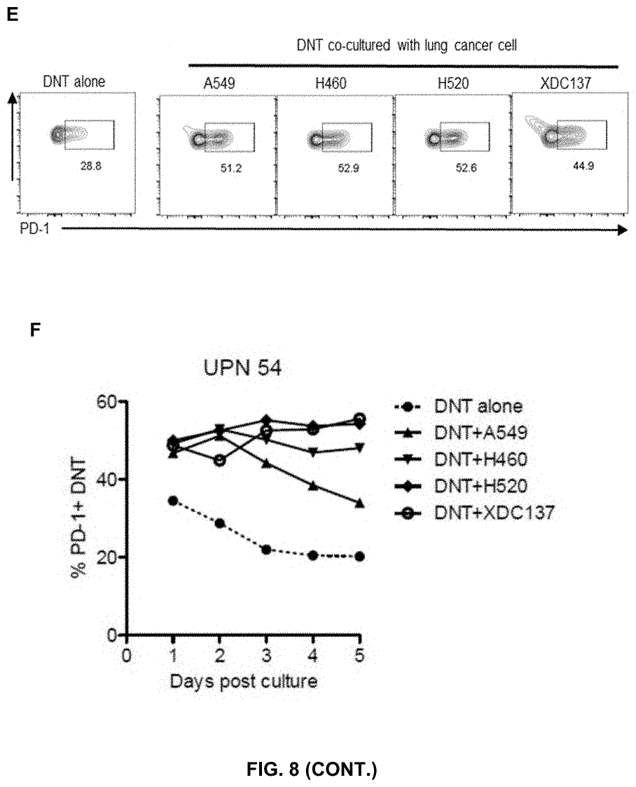

With the observation that significantly fewer DNT cells were found in the patient TILs than in adjacent or normal tissue (10), it was hypothesized that the immunosuppressive tumor microenvironment may prevent DNT cell infiltration. Consistent with this hypothesis, PD-1 was expressed on DNT cells within resected lung tissue, similar to that seen for CD4+ and CD8+ T cells (FIG. 8A). Further, a significantly higher proportion of DNT cells expressed PD-1 within tumors compared to adjacent or normal tissue (CA: 55.5±11.7% vs ADJ: 36.1±14.5% and NOR: 35.5±9.1%). Though tumor-infiltrating DNT cells expressed PD-1, they were the least frequent PD-1+ T cell subset and showed the most variability in PD-1 expression compared to CD4+ and CD8+ T cells (CD4: 65.8±7.1%, CD8: 67.2±7.2%, DNT: 55.5±11.7%, FIG. 8B).

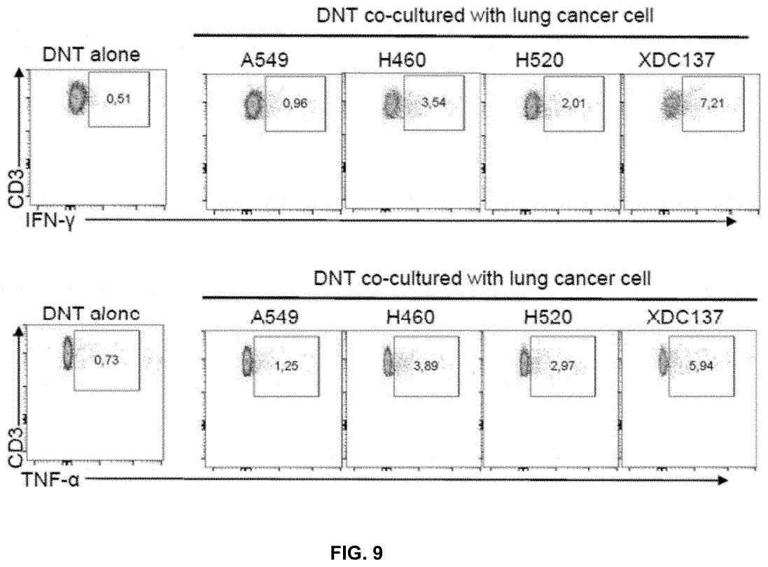

Since patient-derived DNT cells induced a similar level of cytotoxicity against lung cancer cells as those from healthy donors (1, 3), and DNT cells expanded from healthy donors possess features allowing them to be used as an “off-the-shelf” adoptive cell therapy (11), healthy donor DNT cells were utilized to understand the role of PD-1 expression on DNT cells. A similar trend of PD-1 expression for CD8 T cells expanded in this manner was observed. In contrast, CD4 T cells maintained a significantly higher level of PD-1 expression than DNT and CD8 T cells from day 10 until the end of the expansion culture (FIG. 8C). Given that PD-1 expression was higher in tumor infiltrating DNT cells than those in adjacent or normal lung tissues (FIG. 8A), and lung cancer cell lines express different levels of PD-L1 (FIG. 8D), it was determined if co-culture of DNT cells with lung cancer cells was sufficient to induce PD-1 expression. Consistent with the observations in patients, in vitro coculture with 4 different PD-L1+ lung cancer cell lines (A549, H460, H520 and XDC137, FIG. 8D), all resulted in a significant increase in PD-1+ DNT cells when compared with DNT cells cultured alone (FIGS. 8E and F). PD-1 induction was not dependent on the level of PD-L1 expression on lung cancer cells as H520 expressed the lowest level of PD-L1 (FIG. 8D) but induced a similar level of PD1+ DNT cells as H460 which showed a very high level of PD-L1 expression (FIG. 8E). Prolonged co-cultures with lung cancer cells did not further increase PD-1+ DNT cells for any given cell line (FIG. 8F). Co-culture with lung cancer cell lines also increased intracellular expression of IFNγ and TNFα in DNT cells (FIG. 9), suggesting the activation of these T cells by lung cancer cells.

### Anti-PD-1 Treatment Enhances DNT Cell-Mediated Anti-Tumor Activity.

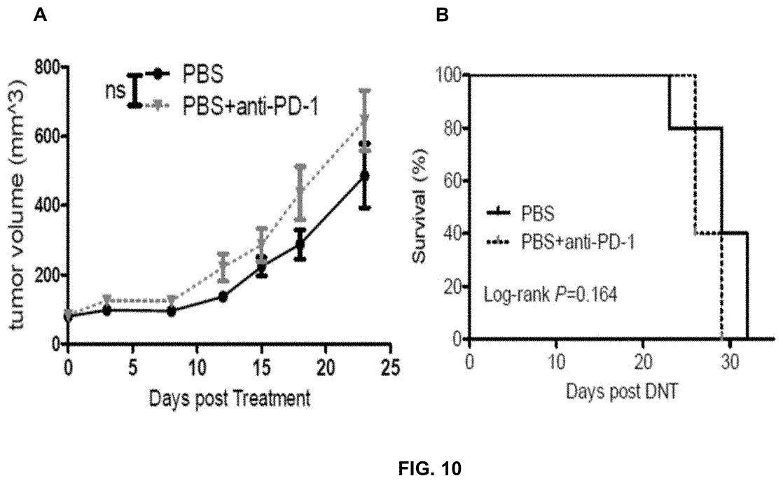

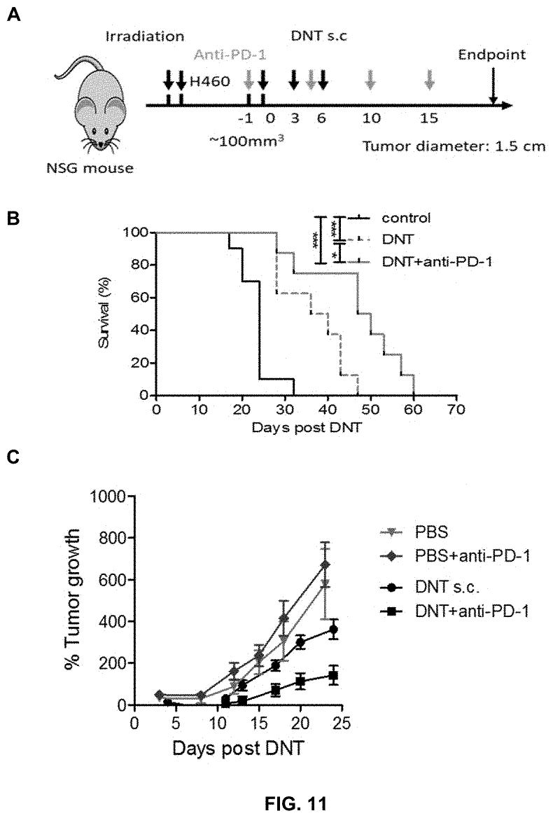

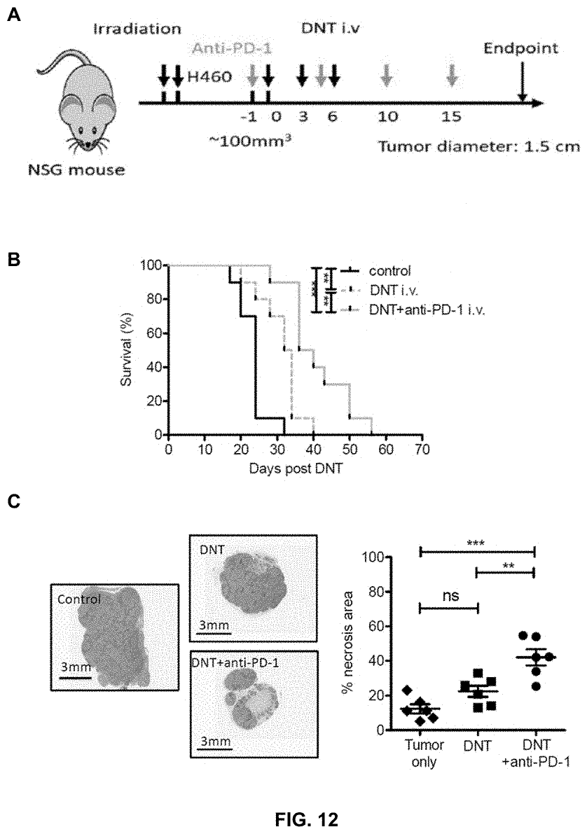

With the propensity of DNT to upregulate PD-1 and cytokines expression in the presence of lung cancer, it was determined if the addition of anti-PD-1 may augment DNT cell-mediated anti-tumor activity in vivo. To observe whether anti-PD-1 can benefit adoptive DNT therapy in vivo, PD-L1 expressing NCI-H460 lung cancer cell line was subcutaneously implanted and established to ˜100 mm³ and DNT cells, with or without anti-PD-1, were administered using two methods, either locally by s.c. peritumoral injection or systemically by intravenous (i.v.) tail vein injection. Anti-PD-1 treatment alone had no effect on tumor growth compared with PBS treated controls (FIG. 10). Peritumoral infusion of DNT cells significantly reduced NCI-H460 tumor volume from 922.1±164.2 mm³ in the control group to 546.5±125.7 mm³ in the DNT cell treated group, resulting in a 40.7±13.6% reduction in tumor volume. Interestingly, the combination of DNT cell injection with anti-PD-1 resulted in an additional 43.1±29.4% reduction of tumor volume (from 546.5±125.7 mm³ in the DNT cell alone treated group to 310.7±160.9 mm³ in the combination group) by day 20. Similarly, systemic i.v. infusion of DNT cells also significantly reduced NCI-H460 tumor volume from 1017.49±246.2 mm³ in the control group to 572.5±186.5 mm³ in the DNT cell treated group, resulting in a 43.7±18.3% reduction in tumor volume, and the combination therapy of i.v. inoculated DNT cells and anti-PD-1 treatment resulted in an additional 32.6±20.0% reduction in tumor volume (from 572.5±186.5 mm³ in the DNT cell alone treated group to 385.9±114.3 mm³ when in combination) by day 20. Importantly, combination therapy prolonged the mean survival time of both s.c. peritumoral inoculated DNT cell treated mice from median 38 days to 48.5 days (FIG. 11B) and anti-PD-1 treatment alone does not alter tumor growth (FIG. 11C). Mice received i.v. inoculated DNT cell as schematically shown in FIG. 12A also increased their mean survival time from median 33 days to 38 days and combination therapy further prolonged survival of recipient mice (FIG. 12B). Similar to what was seen in FIG. 5B, anti-PD-1 significantly increased the proportion of necrotic area detected within tumors from mice receiving combination treatment for i.v. inoculated DNT cells (42.1±10.4% vs 22.4±7.2%; FIG. 12C). These results suggest that DNT cells inhibit tumor growth by actively targeting tumor cells and causing tumor necrosis, and that this activity was enhanced by anti-PD1 therapy. Overall, these results show that addition of anti-PD-1 augments the ability of DNT cells to reduce tumor growth and increase survival of mice.

Anti-PD-1 Treatment Increases DNT Cell Infiltration into Tumor Xenografts

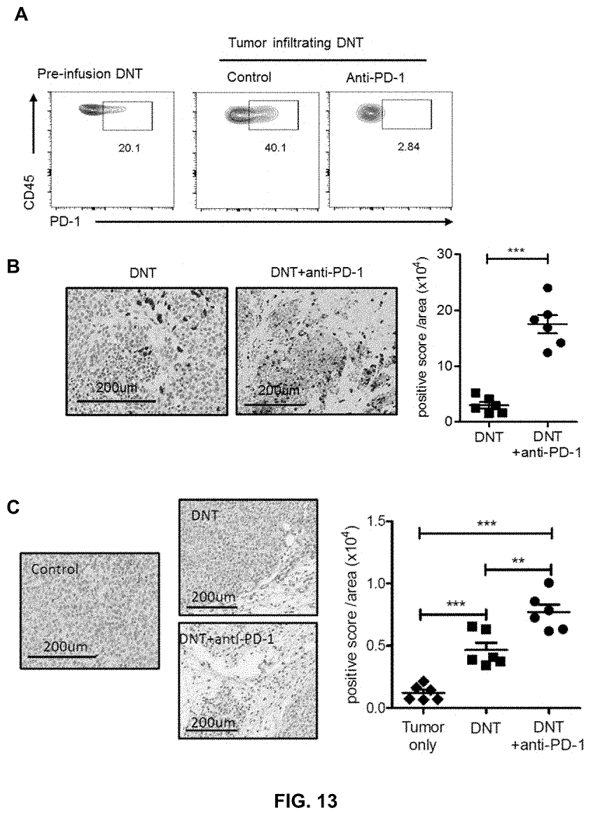

To analyze how anti-PD-1 enhanced DNT cell treatment towards lung cancer xenografts in vivo, tumor infiltrating DNT cells post treatment were analyzed. Consistent with PD-1 induction on DNT cells by lung cancer in vitro, flow cytometric analysis of xenograft infiltrating DNT cells showed a 2-fold increase in PD-1 expression compared to DNT cells prior to infusion (FIG. 13A). Further, anti-PD-1 treatment abrogated PD-1 expression on xenograft infiltrating DNT cells as shown by the lack of staining using anti-PD1 clone EH12.2H7 that recognizes a Nivolumab shared epitope of PD-133,34 (FIG. 13A), suggesting that the Nivolumab treatment effectively blocked the PD-1 epitope on tumor infiltrating DNT cells.

To determine whether anti-PD-1 treatment affects tumor infiltration of DNT cells, DNT cell infiltration of tumor xenografts was quantified by histological analysis. Mice receiving combination treatment of DNT cells and anti-PD-1 antibody had a 5.9±1.2-fold increase in the number of tumor infiltrating DNT cells relative to mice that received DNT cells alone (FIG. 13B). Similarly, i.v. infusion of DNT cells also resulted in a 1.7±0.3-fold increase in DNT cells accumulating in tumor xenografts (FIG. 13C). These data indicate that anti-PD-1 treatment can increase the accumulation of DNT cells in tumor tissue.

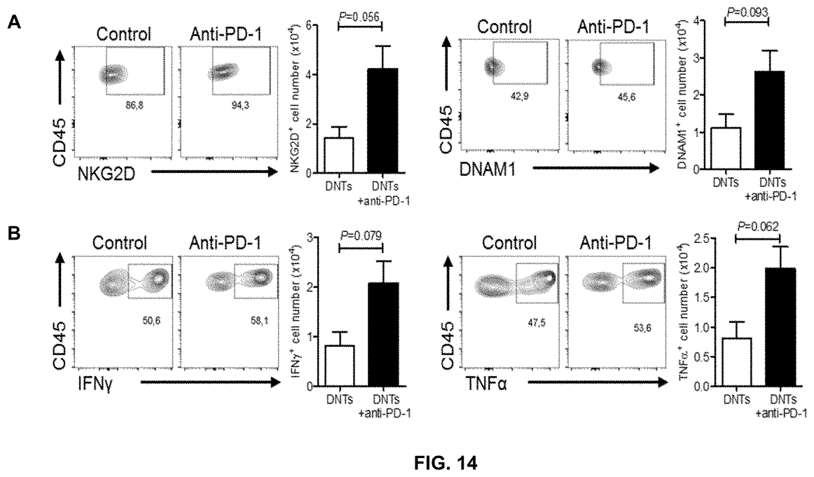

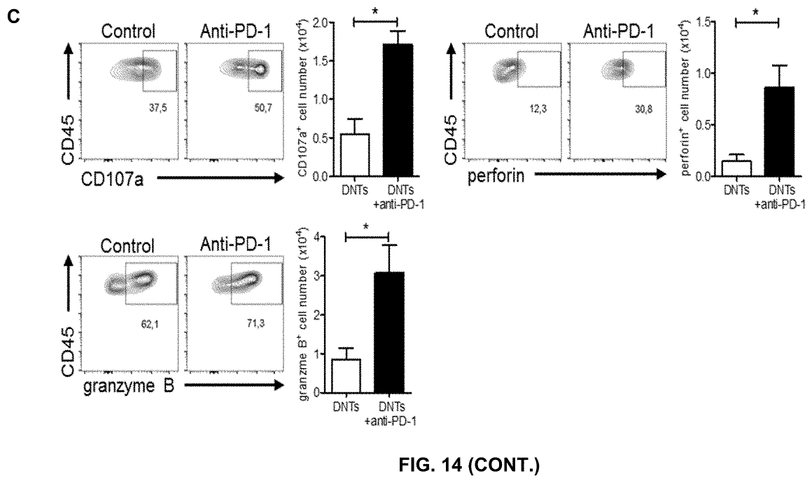

Whether anti-PD-1 treatment could alter the phenotype of tumor infiltrating DNT cells was also investigated. To this end, tumor infiltrating DNT cells were isolated from mice receiving different treatments and expression of cytolytic molecules known to be involved in DNT cell anti-tumor responses were analyzed by flow cytometry. It was found that DNT cells expressing NKG2D and DNAM1 were present in both control and anti-PD-1 treated mice but were more abundant in mice receiving combination therapy than those receiving DNT cells alone, though differences did not reach statistical significance (FIG. 14A). Similarly, mice that received anti-PD-1 showed a greater number of TNFα+ and IFNγ+ DNT cells in the tumor (FIG. 14B). Importantly, consistent with the cytotoxic nature of DNT cells, anti-PD-1 treatment significantly increased the frequency of CD107a+, perforin+, and granzyme B+ DNT cells within tumors (FIG. 14C). These data suggest that anti-PD-1 treatment increases the accumulation of DNT cells within tumors expressing molecules involved in anti-tumor responses.

### Discussion

DNT cell therapy is emerging as a promising adoptive immunotherapy for cancer treatment. Recent data demonstrate that DNTs expanded from healthy volunteers can effectively target AML while sparing normal cells and tissues, including normal lung (1). Infusion of ex vivo expanded human DNTs significantly reduced leukemia load in AML PDX models without any observed toxicity (1). The demonstration of the safety and efficacy of DNTs in preclinical studies has led to the initiation of the first-in-human clinical trial in leukemia patients (NCT03027102).

Unlike conventional T cells which recognize antigens in a TCR-dependent and MHC-restricted fashion, DNTs recognize tumor cells in a TCR-independent manner (1). Consistent with this, it was found that the same lung cancer cells were effectively lysed by DNTs expanded from different donors. Given the heterogeneity of NSCLC, the innate recognition of cancer cells by DNTs may have a greater potential to target a large array of heterogeneic cancers than antigen-specific T cells. This feature would also allow for the use of DNTs from healthy volunteers as a potential “off-the-shelf” cellular therapy.

Collectively, our data show that DNT cells are cytotoxic to lung cancer cells in vitro and in vivo. DNT treatment in combination with anti-PD-1 resulted in increased DNT-mediated anti-tumor activity in vivo. These results highlight the effect of DNT and combinatorial potential of DNT cellular therapy with checkpoint inhibitors for the treatment of solid tumor. These data indicate the potential of DNT cell for the treatment of established lung cancer and that combination of DNT cell with PD-1 blockade may further enhance the treatment efficacy by increasing DNT cell infiltrating to solid tumor.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
NSCLC cell lines have different susceptibilities towards ex vivo expanded DNTs.
Primary and established human NSCLC cell lines were cocultured with ex vivo expanded
DNTs at various E:T ratios. The percentages of specific cytotoxicity against
target cells were detected. E:T EC50 was calculated for each cell line.

| Cell line | % cytotoxicity (Mean ± SD) at different E:T ratios | | | | E:T |
|---|---|---|---|---|---|
| (Cell source) | 20:01 | 10:01 | 5:01 | 2.5:01 | EC50 |
| H2279 (ATCC) | 97.22 ± 12.04 | 87.69 ± 11.65 | 47.69 ± 19.34 | 23.83 ± 13.53 | 4.03 |
| H460 (ATCC) | 89.27 ± 7.23 | 61.52 ± 8.30 | 30.09 ± 12.88 | 14.88 ± 8.75 | 4.86 |
| 12 (PDX) | 83.43 ± 17.97 | 75.32 ± 18.10 | 50.66 ± 21.09 | 24.15 ± 10.97 | 5.76 |
| 178 (PDX) | 83.03 ± 10.55 | 58.96 ± 12.30 | 34.43 ± 7.49 | 11.81 ± 2.83 | 6.7 |
| 426 (PDX) | 83.00 ± 10.50 | 64.54 ± 13.74 | 40.79 ± 7.24 | 16.32 ± 4.46 | 7.47 |
| 277 (PDX) | 81.25 ± 9.27 | 67.62 ± 14.95 | 47.25 ± 8.07 | 21.76 ± 6.73 | 7.56 |
| 655 (PDX) | 81.14 ± 7.99 | 63.21 ± 8.85 | 31.84 ± 1.90 | 17.79 ± 1.77 | 7.97 |
| 229 (PDX) | 80.64 ± 10.41 | 58.96 ± 10.73 | 27.98 ± 14.16 | 10.28 ± 5.43 | 8.86 |
| H125 (ATCC) | 78.62 ± 14.80 | 53.97 ± 19.28 | 24.05 ± 10.39 | 16.39 ± 8.94 | 9.53 |
| 239 (PDX) | 63.57 ± 9.98 | 36.19 ± 16.83 | 11.82 ± 8.76 | 12.45 ± 3.81 | 16.8 |
| A549 (ATCC) | 60.12 ± 10.51 | 35.69 ± 5.41 | 18.28 ± 5.21 | 9.77 ± 4.50 | 16.93 |
| 137 (PDX) | 53.66 ± 15.40 | 30.84 ± 13.30 | 16.67 ± 6.47 | 8.51 ± 2.90 | 20.57 |

TABLE 2
Primary NSCLC cell lines derived from NSCLC PDX models.

| Primary | | Driver | Patient |
|---|---|---|---|
| cell line ID | Histology | mutation* | (Male/Female) |
| 12 | adenocarcinoma (ADC) | BRAF | F |
| 178 | adenocarcinoma (ADC) | no mutation | F |
| 426 | adenocarcinoma (ADC) | not tested | F |
| 277 | adenocarcinoma (ADC) | EGFR | M |
| 655 | adenocarcinoma (ADC) | no mutation | M |
| 229 | adenocarcinoma (ADC) | no mutation | M |
| 239 | adenocarcinoma (ADC) | no mutation | M |
| 137 | adenocarcinoma (ADC) | EGFR | F |

### REFERENCES

1. Lee, J. et al. Allogeneic Human Double Negative T Cells as a Novel Immunotherapy for Acute Myeloid Leukemia and Its Underlying Mechanisms. Clin Cancer Res 24, 370-382 (2018).
2. Zhang, Z.-X., Yang, L., Young, K. J., DuTemple, B. & Zhang, L. Identification of a previously unknown antigen-specific regulatory T cell and its mechanism of suppression. Nature Medicine 6, 782-789 (2000).
3. Merims, S. et al. Anti-leukemia effect of ex vivo expanded DNT cells from AML patients: a potential novel autologous T-cell adoptive immunotherapy. Leukemia 25, 1415-1422 (2011).
4. Joyce, J. A. & Fearon, D. T. T cell exclusion, immune privilege, and the tumor microenvironment. Science 348, 74-80 (2015).
5. Topalian, S. L., Taube, J. M., Anders, R. A. & Pardoll, D. M. Mechanism-driven biomarkers to guide immune checkpoint blockade in cancer therapy. Nat. Rev. Cancer 16, 275-287 (2016).
6. Chen, L. & Han, X. Anti-PD-1/PD-L1 therapy of human cancer: past, present, and future. J Clin Invest 125, 3384-3391 (2015).
7. Herbst, R. S. et al. Predictive correlates of response to the anti-PD-L1 antibody MPDL3280A in cancer patients. Nature 515, 563-567 (2014).
8. June, C. H., O'Connor, R. S., Kawalekar, O. U., Ghassemi, S. & Milone, M. C. CART cell immunotherapy for human cancer. Science 359, 1361-1365 (2018).
9. Newick, K., O'Brien, S., Moon, E. & Albelda, S. M. CAR T Cell Therapy for Solid Tumors. Annu. Rev. Med. 68, 139-152 (2017).
10. Fang L, Ly D, Wang S S, Lee J B, Kang H, Xu H, Yao J, Tsao M S, Liu W, Zhang L. Targeting late-stage non-small cell lung cancer with a combination of DNT cellular therapy and PD-1 checkpoint blockade. J Exp Clin Cancer Res. 38, 123 (2019).
11. Lee J B, Kang H, Fang L, D'Souza C, Adeyi O, Zhang L. Developing Allogeneic Double-Negative T Cells as a Novel Off-the-Shelf Adoptive Cellular therapy for Cancer. Clin Cancer Res. 25, 2241-2253 (2019)

1. A method of treating cancer in a subject in need thereof, the method comprising administering to the subject double negative T cells (DNTs) and a PD-1 or PD-L1 inhibitor.

2. The method of claim 1, comprising administering the DNTs and the PD-1 or PD-L1 inhibitor to the subject at the same time.

3. The method of claim 1, comprising administering the DNTs and the PD-1 or PD-L1 inhibitor to the subject at different times.

4. The method of claim 3, comprising administering the DNTs to the subject within 60 days of administering the PD-1 or PD-L1 inhibitor.

5. The method of claim 3, comprising administering the DNTs to the subject within 30 days of administering the PD-1 or PD-L1 inhibitor.

6. The method of claim 1, comprising administering a first dose and a second dose of DNTs to the subject, wherein the second dose is administered to the subject within 6 months of the first dose.

7. The method of claim 1, comprising administering a first dose and a second dose of the PD-1 or PD-L1 inhibitor to the subject, wherein the second dose is administered to the subject within 6 weeks of the first dose.

8. The method of claim 1, wherein the PD-1 or PD-L1 inhibitor is an antibody or a fragment of antibody such as Fab, F(ab)², or single-chain variable fragment (scFv), a nanobody, or a fusion protein.

9. The method of claim 8, wherein the PD-1 inhibitor is Nivolumab Pembrolizumab, Cemiplimab, Spartalizumab, Tislelizumab, Sintilimab, Toripalimab, Camrelizumab, Dostarlimab, MGA012, or AB122 and the PD-L1 inhibitor is Atezolizumab, Durvalumab, Avelumab, Cosibelimab, Envafolimab or KN035.

10. The method of claim 1, wherein the cancer is lung cancer, melanoma, pancreatic cancer, multiple myeloma, leukemia or lymphoma.

11. The method of claim 10, wherein the lung cancer is non-small cell lung cancer (NSCLC).

12. The method of claim 1, wherein the DNTs express a Chimeric Antigen Receptor (CAR) that preferentially binds to a cancer cell.

13. A method of reducing the growth or proliferation of a tumor, the method comprising contacting the tumor with double negative T cells (DNTs) and a PD-1 or PD-L1 inhibitor.

14. The method of claim 13, wherein the tumor is a solid tumor.

15. The method of claim 13, wherein the PD-1 or PD-L1 inhibitor increases DNT-mediated anti-tumor activity.

16. The method of claim 13, wherein the PD-1 or PD-L1 inhibitor increases DNT tumor infiltration.

17. The method of claim 13, wherein the PD-1 or PD-L1 inhibitor increases DNT-mediated cytotoxicity.

18. The method of claim 13, wherein the tumor is in vivo.

19. A composition comprising double negative T cells (DNTs) and a PD1 or PD-L1 inhibitor.

20. The composition of claim 19, wherein the PD-1 inhibitor is Nivolumab Pembrolizumab, Cemiplimab, Spartalizumab, Tislelizumab, Sintilimab, Toripalimab, Camrelizumab, Dostarlimab, MGA012, or AB122 and the PD-L1 inhibitor is Atezolizumab, Durvalumab, Avelumab, Cosibelimab, Envafolimab or KN035.
